(19) United States
(12) Patent Application Publication
(10) Pub. No.: US 2026/0256602 A1
(43) Pub. Date: Sep. 3, 2026

(54) PROSTHETIC SOCKET

(71) Applicant: OTTOBOCK SE & CO. KGAA, Duderstadt (DE)

(72) Inventor: Lars Benjamin FINKE, Duderstadt (DE)

(21) Appl. No.: 18/718,498

(22) PCT Filed: Dec. 13, 2022
(86) PCT No.: PCT/EP2022/085490

§ 371 Date: Jun. 11, 2024

(30) Foreign Application Priority Data
Dec. 16, 2021 (DE) 10 2021 133 404.7

(51) Int. Cl. A61F 2/80; A61F 2/50; A61F 2/70; A61F 2/76

(52) U.S. Cl. CPC A61F 2/80; A61F 2/70; A61F 2002/502; A61F 2002/7635

(57) ABSTRACT

A prosthetic socket that comprises an open proximal end (4), a distal end (6) with a distal contact surface and a lateral surface that extends between the proximal end (4) and the distal end (6), wherein an effective length of the prosthetic socket extends from the distal contact surface of the distal end (6) to the proximal end (4), wherein an effective circumference of the prosthetic socket extends along the inner surface of the lateral surface, wherein the prosthetic socket comprises an electrical control unit and an adjustment device for adjusting the effective length and the effective circumference, and at least one first sensor (20) for detecting a force acting distally on the amputation stump and at least one second sensor (22) for detecting forces acting radially on the amputation stump, the measured values of which are transmitted to the electrical control unit, the electrical control unit being configured to control the adjustment device according to the measured values of both sensors (20, 22).

The invention relates to a prosthetic socket, which has an open proximal end, a distal end with a distal contact surface and a lateral surface that extends between the proximal end and the distal end, the effective length of the prosthetic socket extending from the distal contact surface of the distal end to the proximal end, an effective circumference of the prosthetic socket extending along the inner surface of the lateral surface.

Such a prosthetic socket is known, for example, from DE 10 2010 019 843 A1 . The prosthetic socket, which is used as part of a prosthesis, has an open proximal end into which an amputation stump is inserted when the prosthesis, of which the prosthetic socket is part, is to be worn by the wearer of the prosthesis. A prosthetic device, such as a knee joint, a lower leg tube or a prosthetic foot or another kind of prosthesis, is arranged at the closed distal end of the prosthetic socket. Prosthetic sockets are used for both lower limb and upper limb prostheses. To achieve particularly effective adhesion of the prosthetic socket to the amputation stump and, where applicable, to achieve a cushioning, the amputation stump is usually surrounded by a liner made from an elastic material, such as silicone.

The amputation stump rests on the distal contact surface of the prosthetic socket, wherein a liner can be located between the amputation stump and the distal contact surface. The distal contact surface is formed by a separate element or component. The distal end is preferably designed to be closed. In this case, the distal contact surface is arranged as a kind of double base parallel to the distal end of the socket. The distal end of the prosthetic socket can also be open.

In the case of lower limb prostheses in particular, such as upper leg prostheses or lower leg prostheses, the prosthesis bears the entire weight of the wearer of the prosthesis with every step they walk or run and as such is subjected to high mechanical loads. The same applies for the amputation stump inside the prosthetic socket. It is therefore very important that the prosthetic socket is especially well adapted to the geometric shape of the amputation stump in order to avoid pressure points and therefore wounds and pain. The problem here is that the shape of the amputation stump is subject to change, particularly in the initial period after amputation, as the muscles in the amputation stump in particular regress due to inactivity and scars and tissue hardening develop. The stump volume also changes over the course of a day due to redistribution of fluids in the tissue.

In order to take these changes in the volume of the amputation stump and its shape into account, it is known in the prior art to render both the effective circumference and the effective length of the prosthetic socket adjustable. To this end, DE 10 2010 019 843 A1 comprises a tensioning device which, for example, has a tensioning cord or a wire by means of which a tension can be applied to the various elements of the prosthetic socket. As a result, the effective circumference of the prosthetic socket can be adjusted by the user of the prosthetic socket themselves. In addition, the socket described in said publication has an adjustment device, by means of which the effective length of the prosthetic socket, also known as a sleeve in the prior art, can be adjusted.

The disadvantage is that, despite the fact that the effective length and the effective circumference are adjustable, the fit of the prosthetic socket cannot be optimally adjusted by the wearer of the respective prosthetic, meaning that pain and issues may occur. The user and wearer of a prosthesis often cannot recognize, at least easily, whether a decrease in volume of the amputation stump should be accounted for by reducing the effective circumference, reducing the effective length or both. The same applies for an increase in volume of the amputation stump, as happens over the course of a day. If the wearer of the prosthesis increases the effective circumference of the prosthetic socket, this may counter the high pressure acting on the amputation stump caused by the increase in volume and provide relief. At the same time, however, this may mean that the distribution of weight and forces within the prosthetic socket is no longer optimal, possibly resulting in pressure points and pain. Conversely, if the effective length of the prosthetic socket is increased too much, the distal end of the amputation stump may lose contact with the inner surface of the distal end of the prosthetic socket. This can also change the distribution of forces within the system of prosthetic socket and amputation stump, resulting in end of the amputation stump.

The invention is therefore based on the task of eliminating or at least reducing the disadvantages of the prior art.

The invention solves the addressed task by way of a prosthetic socket according to the preamble of claim 1, which is characterized in that the prosthetic socket comprises an electrical control unit and an adjustment device for adjusting the effective length and the effective circumference, and at least one first sensor for detecting a force acting distally on the amputation stump and at least one second sensor for detecting forces acting radially on the amputation stump, the measured values of which are transmitted to the electrical control unit, the electrical control unit being configured to control the adjustment device according to the measured values of both sensors.

The prosthetic socket according to the invention has an adjustment device, with which the effective length and the effective circumference of the prosthetic socket can be set and changed. The adjustment device is controlled via the electrical control unit of the prosthetic socket, which may be an electronic data processing device, for example. This preferably allows the effective circumference and the effective length to be adjusted to the individual needs of the prosthesis wearer and the current situation of the amputation stump.

The prosthesis socket has one first sensor for detecting a force acting distally on the amputation stump and at least one second sensor for detecting forces acting radially on the amputation stump. Preferably, the first sensor and/or the second sensor comprises a pressure sensor, a force sensor, a torque sensor and/or a deformation sensor. In the context of the present application, a pressure sensor may detect a pressure when conclusions can be drawn about the prevailing pressure from the measured values of the pressure sensor. It is not necessary for the sensor to directly measure the pressure. Correspondingly, a force sensor is understood to mean any sensor whose measured values can be used to draw conclusions about a force. Within the scope of the present invention, a torque sensor is any sensor whose measured values enable conclusions about a torque and a deformation sensor is any sensor whose measured values can be used to make statements on a deformation of the amputation stump.

Systems that work hydraulically or pneumatically, for example, can be used as a first sensor and/or second sensor. In a hydraulic or pneumatic system, for example, the pressure of the hydraulic fluid or the gas in the hydraulic or pneumatic system is measured. It is a measure of the pressure applied to the system from outside, for example by the amputation stump and/or the prosthetic socket. The working medium of a pneumatic system is preferably a compressible gas; the working medium of a hydraulic system is preferably an incompressible fluid, such as a liquid, preferably a hydraulic oil.

An optical sensor, for example, can be used as a first and/or second sensor, said optical sensor measuring the acting pressure by means of an optical measurement. For example, a pressure-sensitive element, such as a spring element, can be used for this purpose, which is expanded or compressed to a greater or lesser extent by the amputation stump or is deformed or deflected in some other way. This expansion, compression, deformation or deflection can then be detected by the optical sensor.

For example, a deformation sensor can be a capacitive and/or resistive sensor. In the case of a capacitive sensor, for example, the outside of the amputation stump or a liner pulled over it on the one hand and the inner surface of the prosthesis socket on the other form the two plates of an electrical capacitor. The distance between the two plates formed in this way determines the capacitance of the capacitor, which is measured. A change in distance and therefore a deformation of the amputation stump changes the capacitance of the capacitor. The distance can thus be indirectly determined via the capacitance of the capacitor.

With a resistive sensor, a deformation of the amputation stump changes an electrical resistance, which is measured. Consequently, a change in the resistance contains information about a deformation of the amputation stump.

Alternatively or additionally, an expansion measuring strip or a Hall sensor can be used as a first and/or second sensor to detect a deformation of the amputation stump.

Alternatively or additionally, an expansion measuring strip or sensor can be used as a first sensor and/or a second sensor to detect the deformation of part of the adjustment device of the prosthetic socket. Furthermore, an expansion measuring strip can be used in combination with a load cell to detect a force applied to the stump.

Irrespective of the choice of sensors, it is important that a first sensor and a second sensor are used whose measured values are used to control the adjust device.

The first sensor and the second sensor send their measured values to the electrical control unit in the form of measurement signals. Said control unit is configured to determine whether and, if so, how the effective length and the effective circumference of the prosthetic socket must be adjusted depending on these measured values. In addition, the electrical control unit is configured to generate control signals and to transmit them to the adjustment device. The adjustment device is controlled by the control signals in such a way that the effective length and the effective circumference are adjusted accordingly.

In the case of a prosthetic socket according to the invention, it is not absolutely necessary for the effective length and the effective circumference to always be changed simultaneously. It is quite possible that for certain situations the electrical control unit recognizes from the measured values transmitted to it that only one of the two, for example the effective circumference or the effective length, needs to be changed. Regardless of whether the effective length or the effective circumference or both variables are to be changed, both the detected distal pressure and the detected radial pressure are input variables, on the basis of which the electrical control unit performs its algorithms and processes and generates the control signals. In the case of a prosthetic socket according to the invention, it is not possible that the electrical control unit controls the adjustment device without both the distal pressure and the radial pressure being detected and transmitted to the electrical control unit.

Preferably, the adjustment device has a moveable end element, by means of which the effective length of the prosthetic socket can be changed, and/or at least one moveable circumferential element, by means of which the effective circumference can be changed. The moveable end element then preferably forms the distal contact surface.

In a preferred embodiment, the change in the effective length and the effective circumference effects a change in the distal pressure and the radial pressure, which leads to a change in the measured values of both sensors. This allows a control loop to be generated. The electrical control unit preferably accesses an electronic memory where target values for the measured values of both sensors are stored, preferably for the distal pressure and the radial pressure. The electrical control unit is configured to process said stored target value, such as a stored distal pressure and radial pressure, and the measured values of the sensors in such a way that they can be compared to each other so that, on the basis of this comparison, the electrical control unit determines whether and in which direction the effective circumference and the effective length have to be changed.

Preferably, the moveable end element forms the distal contact surface of the distal end of the prosthetic socket. In order to change the effective length of the prosthetic socket, in this case the moveable end element is moved towards or away from the open proximal end of the prosthetic socket. In order to reduce the effective length of the prosthetic socket, the moveable end element is moved towards the proximal end of the prosthetic socket. A movement in the opposite direction, i.e. away from the the prosthetic socket.

The at least one moveable circumferential element is preferably arranged in such a way that it forms at least one part of the inner surface of the lateral surface of the prosthetic socket. Particularly preferably, the at least one moveable circumferential element is arranged in a recess of the base body of the prosthetic socket. In this case, the effective length of the prosthetic socket can be reduced especially easily by moving the at least one moveable circumferential element in the recess of the base body of the prosthetic socket radially inwards, i.e. towards the longitudinal axis of the prosthetic socket along which the effective length of the prosthetic socket also extends. To increase the effective circumference of the prosthetic socket, the at least one moveable circumferential element in this configuration is moved in the opposite direction, i.e. radially outwards in relation to the longitudinal axis of the prosthetic socket.

Alternatively or additionally, the prosthetic socket has multiple circumferential elements that overlap each other and that together form at least part of the effective circumference, but preferably the entire effective circumference, of the prosthetic socket. This design is often referred to as a “tulip”. In this case, the prosthetic socket preferably has a tensioning device, for example in the form of a tensible wire or cable, by means of which the degree of overlap of the various circumferential elements can be adjusted and changed. If the effective circumference of the prosthetic socket in this embodiment is to be reduced, the tension on the wire or cable is increased and the circumferential elements, which are designed to be moveable, moved in such a way that they overlap to a greater degree. The effective circumference of the prosthetic socket decreases as a result. Conversely, a reduction in the tension on the wire or cable can cause the moveable circumferential elements to move in such a way that they overlap to a lesser degree, thereby increasing the effective circumference of the prosthetic socket. Of course, a combination of the various configurations, for example multiple overlapping circumferential elements and at least one circumferential element positioned in a recess of the base body, is also possible.

The prosthetic socket preferably has a drive that moves the moveable end element. The prosthetic socket has at least one drive that moves the at least one moveable circumferential element. In one possible embodiment, the movements are carried out by the same drive, so the prosthetic socket only has a single drive. The drive or drives form part of the adjustment device.

In a preferred embodiment of the prosthetic socket, the first sensor detects a force exerted on the moveable end element by an amputation stump inserted into the prosthetic socket. The second sensor detects a force exerted on the moveable circumferential element by an amputation stump inserted into the prosthetic socket. The electrical control unit, preferably an electronic data processing device, is configured to move the adjustment device, i.e. the moveable end element, for example, and/or the at least one moveable circumferential element depending on the measured values of the sensors. Particularly preferably, the values detected by the sensors are each compared to a target value or a target value range in the electrical control unit.

The target values stored in the electrical control unit may contain both absolute values and target ratios of the detected forces on the amputation stump to each other. The aim is to achieve the most even pressure distribution possible from the prosthesis socket to the amputation stump. However, this is subject to user-specific individual fluctuations and is preferably determined and tested for each patient.

For example, if a force detected on one of the moveable elements is greater than a predetermined target value, the electrical control unit moves the moveable element away from the amputation stump to reduce the pressure load. For example, if a force detected on one of the moveable elements is smaller than a predetermined target value, the electrical control unit moves the moveable element in such a way that the pressure increases. To this end, the respective moveable element is moved, for example, towards the amputation stump. Particularly preferably, both the moveable end element and the at least one moveable circumferential element are moved simultaneously. However, it is potentially sufficient to move only the end element or the circumferential element in order to achieve the desired pressure distribution. For example, the electrical control system sends control signals to the drive, such as an electric motor, which moves the respective element, i.e. the end element and/or the circumferential element.

If the detected pressure on both pressure sensors is, for example, smaller than the predetermined target value for the respective sensor, the electrical control unit moves the moveable end element and/or the at least one moveable circumferential element so that the volume within the prosthetic socket decreases. If, however, the detected pressure on both pressure sensors is greater than the predetermined target value for the respective sensor, the electrical control unit moves the moveable end element and/or the at least one moveable circumferential element so that the volume within the prosthetic socket increases. The range between the first target value and the predetermined second target value can be referred to and considered the target value range.

A coupling of the two movements of the moveable end element and the moveable circumferential element is preferably achieved by using a mechatronic control system, which includes the electrical control system and the at least one drive.

Particularly preferably, the prosthetic socket has at least one additional sensor, preferably a moisture sensor, a temperature sensor and/or an oxygen saturation sensor, and the electrical control unit is configured to move the moveable end element and/or the moveable circumferential element depending on the measured values of the first and second sensor as well as of this at least one additional sensor. Preferably, the moisture sensor is arranged in such a way that it measures the moisture between the prosthetic socket and the amputation stump, preferably between the prosthetic socket and a liner in which the amputation stump is located. If the additional sensor is a temperature sensor, it is preferably arranged in such a way that the temperature inside the prosthetic socket is measured, i.e. in the space between prosthetic socket and amputation stump. An oxygen saturation sensor is preferably arranged to be able to determine the oxygen saturation of the blood within the amputation stump.

Preferably, the at least one additional sensor is configured to detect sensor data from which conclusions can be drawn about the state of movement of the wearer of the prosthetic socket. Particularly preferably, the at least one additional sensor has at least one inertial sensor that is preferably configured to determine a spatial position, a speed and/or a change in the angle of the prosthetic socket or of a component directly or indirectly connected to the prosthetic socket. For example, the electrical control unit can control the adjustment device in such a way that the effective length and/or the effective circumference of the prosthetic socket increase(s) when the wearer of the prosthetic socket is sitting down. In this case, no or at least no regularly repeated movements of the prosthetic socket are detected. The prosthesis socket is also not subjected to as much or even any stress.

In the following, an embodiment example of the invention will be explained in more detail with the aid of the accompanying figures. They show

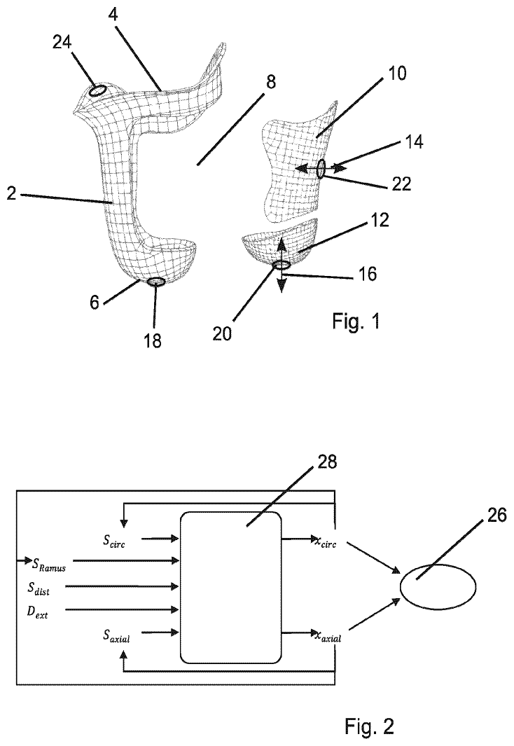

FIG. 1—the schematic representation of various parts of a prosthetic socket and

FIG. 2—a schematic flow chart that illustrates how the electrical control unit functions.

FIG. 1 schematically depicts various components of a prosthetic socket according to a first embodiment example of the present invention. The prosthetic socket has a base body 2, which comprises an open proximal end 4 and a distal end 6, which is closed in the embodiment example shown. It also has a recess 8 in which a circumferential element 10 is arranged that can be moved relative to the base body 2. At the distal end 6 of the base body 2 is a moveable end element 12, which is depicted next to the base body 2 in FIG. 1. The moveable circumferential element 10 can be moved along the radial double arrow 14 and the moveable end element 12 along the axial double arrow 16. The terms “axial” and “radial” refer to the longitudinal extension of the prosthetic socket, which extends along the effective length of the prosthetic socket from the inner surface of the distal end 6 to the proximal end 4.

In the embodiment example shown, a distal sensor 18 is arranged at the distal end 6 of the base body 2. Said sensor measures an axial pressure or an axial force. The latter is referred to as a distal force and is the resulting overall force on a socket adapter, which is not depicted in FIG. 1 for clarity. Further prosthetic elements may be arranged on the socket adapter, for example an upper leg prosthesis, such as a prosthetic knee joint, in the embodiment example shown. A first sensor 20 is positioned on the moveable end element 12 and measures the pressure or force exerted on the distal end of an amputation stump. This pressure is referred to as axial pressure. The prosthetic socket has a second sensor 22 which, in the embodiment example shown, is arranged on the moveable circumferential element and measures a radial force or radial pressure. A ramus pressure sensor 24 measures the force of a bony contact of the prosthesis socket and is also referred to as a ramus sensor.

FIG. 2 schematically demonstrates how the individual data or measured values recorded by the individual sensors can be used. The aim is to optimize the selection of the effective length xₐₓᵢₐₗ and the effective circumference x_{circ}, which can be changed and adapted by displacing the moveable end element 12 and the moveable circumferential element 10. These form the target configuration 26. To this end, the measured values S_{circ} of the second pressure sensor 22, S_{Ramus} of the ramus pressure sensor 24, S_{dist} of the distal pressure sensor 18 and Sₐₓᵢₐₗ of the first pressure sensor 20 are fed to an electrical control unit 28. In the embodiment example shown, additional data Dₑₓₜ, for example from external sensors not depicted in FIG. 1, and/or manual user specifications, for example, are taken into account and likewise fed to the electrical control unit 28.

The electrical control unit 28 uses the data and measured values provided to calculate values for the effective circumference x_{circ} and the effective length xₐₓᵢₐₗ Of the prosthetic socket and controls a drive for the respective adjustment device in sensors and therefore the input values provided to the electrical control unit 28 change until the target configuration 26, which contains target values for the individual measured variables for example, is achieved.

### Reference List

2 base body
4 proximal end
6 distal end
8 recess
10 circumferential element
12 end element
14 radial double arrow
16 axial double arrow
18 distal sensor
20 first sensor
22 second sensor
24 ramus pressure sensor
26 target configuration
28 electrical control unit

1. A prosthetic socket, comprising:
an open proximal end;
a distal end with a distal contact surface;
a lateral surface that extends between the proximal end and the distal end,
wherein an effective length of the prosthetic socket extends from the distal contact surface of the distal end to the proximal end,
wherein an effective circumference of the prosthetic socket extends along an inner surface of the lateral surface;
an electrical control unit and an adjustment device for adjusting the effective length and the effective circumference;
at least one first sensor for detecting a force acting distally on an amputation stump; and
at least one second sensor for detecting forces acting radially on the amputation stump,
wherein the at least one first sensor and the at least one second sensor are configured for transmitting measured values to the electrical control unit, wherein the electrical control unit is configured to control the adjustment device according to the measured values of the at least one first sensor and the at least one second sensor.

2. The prosthetic socket according to claim 1, wherein the at least one first sensor and/or the at least one second sensor comprises a pressure sensor, a force sensor, a torque sensor, and/or a deformation sensor.

3. The prosthetic socket according to claim 1 wherein the adjustment device comprises a moveable end element for changing the effective length and/or at least one moveable circumferential element for changing the effective circumference.

4. The prosthetic socket according to claim 3, wherein the adjustment device comprises the moveable end element and wherein the moveable end element forms the distal contact surface of the distal end.

5. The prosthetic socket according to claim 3 wherein the adjustment device comprises the at least one moveable circumferential element and wherein the at least one moveable circumferential element forms at least part of the inner surface of the lateral surface.

6. The prosthetic socket according to claim 1 further comprising a base body, and wherein the at least one circumferential element is arranged in a recess of the base body.

7. The prosthetic socket according to claim 1 wherein the at least one first sensor comprises a first pressure sensor or wherein the at least one second sensor comprises a second pressure sensor, and wherein
the first pressure sensor is configured and arranged to measure a pressure exerted on the moveable end element by an amputation stump inserted into the prosthetic socket, and/or
the second pressure sensor is configured and arranged to measure a pressure exerted on the at least one moveable circumferential element by an amputation stump inserted into the prosthetic socket.

8. The prosthetic socket according to claim 7, further comprising at least one additional sensor, and
wherein the electrical control unit is configured to move the moveable end element and/or the at least one moveable circumferential element depending on measured values of the at least one additional sensor.

9. The prosthetic socket according to claim 8, wherein the at least one additional sensor is configured to detect sensor data which are relatable to a state of movement of a wearer of the prosthetic socket.

10. The prosthetic socket according to claim 9, wherein the at least one additional sensor comprises at least one inertial sensor configured to determine
a spatial position,
a speed, and/or
a change in the angle
of the prosthetic socket or of a component of the prosthetic socket directly or indirectly connected to the prosthetic socket.

11. The prosthetic socket according to claim 8 wherein the at least one additional sensor is selected from the group consisting of a moisture sensor, a temperature sensor, and an oxygen saturation sensor.
